(19) United States
(12) Patent Application Publication
(10) Pub. No.: US 2026/0013780 A1
(43) Pub. Date: Jan. 15, 2026

(54) Method and Apparatus for Assessing Electrocortical Consequences of Brain Injury

(71) Applicants: WISCONSIN ALUMNI RESEARCH FOUNDATION, Madison, WI (US); University of Milan, Milan (IT); Tiny Blue Dot, Inc., Santa Monica, CA (US)

(72) Inventors: Giulio Tononi, Verona, WI (US); Marcello Massimini, Milan (IT); Simone Sarasso, Milan (IT); Mario Rosanova, Milan (IT); Christof Koch, Pasadena, CA (US)

(21) Appl. No.: 18/992,109

(22) PCT Filed: Jun. 30, 2023
(86) PCT No.: PCT/US2023/026681

§ 371 Date: Jan. 7, 2025

Related U.S. Application Data
(60) Provisional application No. 63/367,957, filed on Jul. 8, 2022.

(51) Int. Cl. A61B 5/377; A61B 5/00; A61B 5/291; A61B 5/374

(52) U.S. Cl. CPC A61B 5/377; A61B 5/291; A61B 5/374; A61B 5/6814

(57) ABSTRACT

An evaluation of suppressed neural function around the site of a brain lesion or injury is provided by repeated stimulations of the brain, for example, using transcranial magnetic stimulation, at different locations around the lesion and monitoring time and frequency domain characteristics of the resulting EEG signals to identify cortical bistability associated with functional loss that could subsequently be targeted for neural rehabilitation.

### CROSS-REFERENCE TO RELATED APPLICATIONS

This application claims the benefit of U.S. provisional application 63/367,957 filed Jul. 8, 2022 and hereby incorporated by reference.

### BACKGROUND OF THE INVENTION

The present invention relates to techniques for assessing brain injuries and, in particular, to a method of assessing cortical bistability around a brain lesion or other injury such as can guide rehabilitative efforts.

Focal brain lesions, including stroke and traumatic brain injury, are the main causes of disability and loss of productivity in the world. Physicians currently diagnose and treat these conditions as if they were caused merely by local dysfunction due to the pathological loss of brain tissue at the injury site. However, there is growing evidence that clinical symptoms reflect widespread functional alterations in cortical areas extending well beyond the area of brain tissue injury or loss.

These functional alterations outside of the primary injury can potentially be treated by re-normalizing patterns of electrical activity through “circuit-based” neuromodulation and targeted pharmacological intervention. This ideal therapeutic approach, however, is currently limited by lack of a reliable understanding of the neuronal alterations that need to be corrected.

Electrophysiological evidence derived from animal models and non-invasive clinical recordings in anoxic, stroke and traumatic brain-injured subjects show a persistent and relative slowing of electroencephalographic (EEG) and magnetoencephalographic (MEG) rhythms in areas ipsilateral to the lesion and possibly extending to the contralateral cerebral hemisphere. More recently, human intracranial recordings have shown that post-lesion intracerebral activity during wakefulness can be characterized by slow waves, associated with silent neuronal periods (OFF-periods) closely matching those recorded during baseline deep sleep in the same individuals. Sleep-like slow waves and OFF-periods are prominent in the perilesional areas but can also percolate through a network of connected areas. The pathological intrusion of local slow waves in the awake brain and the associated neuronal bistability between ON-and OFF-periods are critical from a clinical standpoint as this phenomenon, called “local sleep” in wake, leads to negative cognitive and behavioral consequences.

### SUMMARY OF THE INVENTION

The present invention provides a way of probing the brain with non-invasive electrical, magnetic, ultrasonic, or other mode of brain stimulation to identify regions in the vicinity of the lesion or injury that may have been indirectly affected by the injury, allowing better assessment of the injury and evaluation of treatment options. Generally, the localized area of stimulation is scanned through different locations around the injury site and the response to that stimulation is measured to detect cortical bistability. The result can provide a mapping of circuit function outside of the injury area to better assess and treat patients with brain injury.

One embodiment of the invention provides an apparatus for assessing brain injury including an electroencephalographic sensor system collecting electrical signals from electrically active neurons within a patient's brain and a neural stimulator for triggering a localized stimulation of neurons within the patient's brain at a set of different location and times using, for example, either electrical, magnetic, or ultrasonic stimulation. An electronic computer receives the electrical signals from these electroencephalographic sensors and executes a stored program to: (i) monitor electrical signals from the electroencephalographic sensor responding to localized stimulations at the different locations and times from firing neurons; (ii) determine a cortical bistability value at the different locations and times; and (iii) output a map providing information about cortical bistability by linking the determined cortical bistability to different neocortical regions according to their locations.

It is thus a feature of at least one embodiment of the invention to provide better insight into ancillary injury of neocortex around a lesion or the like.

The apparatus may include a position tracker monitoring the three-dimensional (3-D) location of the neural stimulator to provide a 3-D location value of the localized brain stimulation to the electronic computer, and the location of value may be linked to a corresponding electrical signal and determination of cortical bistability to provide the map.

It is thus a feature of at least one embodiment of the invention to provide a simplified apparatus that may use a position tracker and standard stimulation techniques.

The electronic computer may further execute the stored program to receive radiological or magnetic resolution imagery (MRI) pertaining to physiological injury data locating the injury site in the brain and wherein the map provides an indication of the injury site.

It is thus a feature of at least one embodiment of the invention to permit visualization of the injury site both to guide the stimulation and to provide context for the regions of cortical bistability.

The electronic computer may determine cortical bistability by determining high-frequency activity, where reduced high-frequency activity is indicative of increased cortical bistability.

It is thus a feature of at least one embodiment of the invention to provide a simple proxy for cortical bistability amenable to multiple samplings over an area to define a map.

The determination of high-frequency activity may evaluate a peak spectral power.

It is thus a feature of at least one embodiment of the invention to simply identify the slow wave activity associated with cortical bistability.

The electronic computer may further determine cortical bistability by assessing a phase relationship between the onset of brain stimulation and a time of electrical signals following the brain stimulation.

It is thus a feature of at least one embodiment of the invention to reduce the effects of random noise and neural activity by establishing causality between the stimulation and received signal.

The phase relationship may be evaluated in electrical signals band limited to more than 4 Hz and less than 40 Hz in frequency content.

It is thus a feature of at least one embodiment of the invention to use a frequency band-limited signal in a range affected by cortical bistability to improve the sensitivity of the measurement.

The phase relationship may be established by evaluating an inter-trial coherence in electrical signals associated with multiple trials of brain stimulation.

It is thus a feature of at least one embodiment of the invention to provide a robust and well-characterized method of assessing phase correlation.

The evaluation of cortical bistability values may be determined using the electrical signals only in a time window of less than 500 ms following brain stimulation.

It is thus a feature of at least one embodiment of the invention to further improve the sensitivity of the measurement by rejecting signals outside of an expected time-window of reaction to the stimulation.

The electroencephalographic sensor system may employ cutaneous electrodes placed on the patient and the neural stimulator may be a transcranial magnetic stimulation (TMS) device.

It is thus a feature of at least one embodiment of the invention to provide a noninvasive assessment of brain injury.

The map may be an image representation of the brain with regions of cortical bistability having predetermined shading corresponding to the degree of bistability.

It is thus a feature of at least one embodiment of the invention to provide an intuitive understanding of neuronal health in cortical areas strongly connected to the lesion (peri-lesional), or for example, to assist with targeted rehabilitation of the patient.

Other features and advantages of the invention will become apparent to those skilled in the art upon review of the following detailed description, claims and drawings in which like numerals are used to designate like features.

### BRIEF DESCRIPTION OF THE DRAWINGS

FIG. 1 is a simplified block diagram of an apparatus implementing the present invention including a transcranial magnetic stimulation (TMS) coil positionable at various locations around the patient's head as coordinated by a tracking system and an electroencephalographic (EEG) system for detecting EEG signals in response to a stimulation, both communicating with a controller executing a stored program;

FIG. 2 is a flowchart showing the steps of the present invention executed in part by the controller;

FIG. 3 is an example output image characterizing a lesion and tissue outside of the lesion subject to cortical bistability;

FIG. 4 is a simplified representation of a power spectrum of the signal collected by the electroencephalographic system for assessing high-frequency activity used to identify neurons subject to cortical bistability; and

FIG. 5 is a simplified representation of two successive acquisitions (trials) of EEG signals showing assessment of time coherence of the acquired EEG signals with respect to the stimulation.

### DETAILED DESCRIPTION OF THE PREFERRED EMBODIMENT

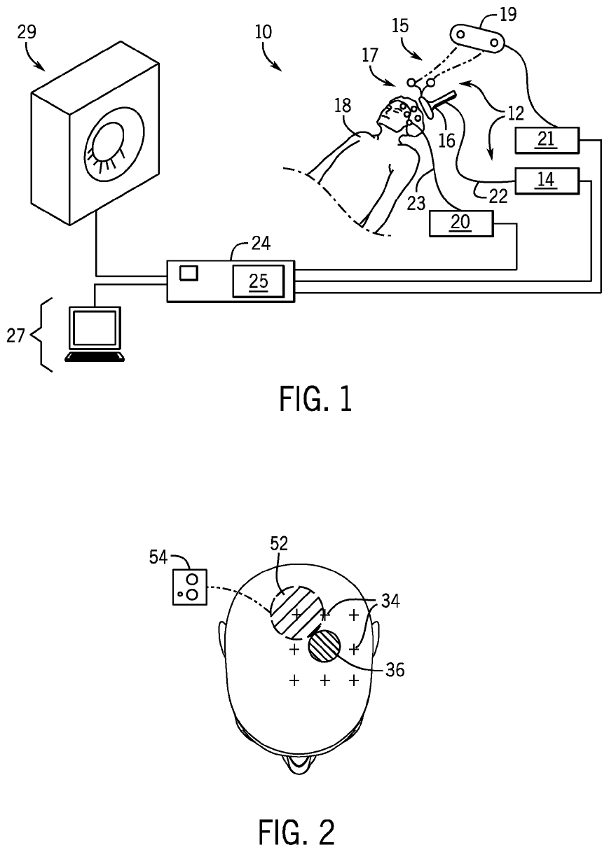

Referring now to FIG. 1, an embodiment of the apparatus 10 of the present invention may use a transcranial magnetic stimulation (TMS) device 12 having a power unit 14 and a coil 16. During operation of the TMS device 12, a set of capacitors or other energy storage devices in the power unit 14 are charged and then rapidly connected to the coil 16 by leads 22 creating a monophasic or biphasic pulse of current in the coil 16 positioned at the top of the head of an awake patient 18.

As is generally understood in the art, the pulse of current so produced in the coil 16 causes a rapidly changing magnetic flux in the brain of the patient 18 which in turn induces an electrical current within the brain tissue that stimulates neuron activity. The coil 16, in one embodiment, may be a “butterfly coil” having windings in a figure-eight pattern to provide opposed magnetic flux in adjacent loops focusing the flux to a compact region. Other coils such as single loop coils and the like may also be used. A TMS device 12 suitable for use with the present invention is commercially available, for example, from Nexstim of Helsinki, Finland, under the trade name Nexstim NBS. Alternately, transcranial electrical stimulation (TES) or ultrasound may be used.

The coil 16 may operate in conjunction with a position tracking system 15 providing a tracking appendage 17 attached to the coil 16 and a monitor unit 19, for example, tracking the appendage 17 (and hence the coil 16) using infrared imaging. A tracking system suitable for use with this invention is commercially available under the trade name Nexstim of Helsinki, Finland, under the trade name Nexstim NBS. Generally, the position tracking system 15 generates a quantitative position and orientation of the coil 16 in 3D space in real-time relative to the patient who may be stabilized in a fixed location with respect to the position tracking system 15 or may be associated with a similar tracking appendage so as to determine only relative motion between the patient 18 and the coil 16.

In an alternative embodiment, the coil 16 may be held in a robotic manipulator (not shown) for scanning in a desired pattern as will be described below.

The TMS device 12 may be controlled by a controller 24, for example, an electronic computer executing a stored program 25 held in computer memory, and communicates with a user terminal 27 allowing for the output of a brain map on a graphical display, as will be described, and other data and the input of commands on a keyboard or similar device according to methods well known in the art. In this regard, the controller 24 controls the timing of the application for stimulating pulse through the coil 16 or may receive timing data indicating that timing. In addition, the controller 24 may also receive position and location information from the position processor 21 and may provide instructions to a human operator with respect to desired positioning of the coil 16 or may provide instructions to a robotic manipulator for such positioning as discussed above. A manipulator suitable for this purpose is commercially available under the trade name of Axilum TMS Cobot or Axilum TMS Robot from Brainbox LTD cited above.

The controller 24 may also receive EEG signals from an EEG processor 20, the latter communicating with the patient 18 over leads 23 by means of a set of cutaneous electrodes 26 placed on the patient's head, for example, using an electrode cap having in excess of 60 electrodes for wide area coverage, however, the invention may also be used with fewer electrodes. The EEG processor 20 may provide standard EEG amplifiers, gating circuitry, and filters to provide for continuous acquisition of EEG signals without disruption by the pulse produced by the TMS device 12. The EEG device is either endowed with a wide dynamic range coupled with a high sampling rate or with a sample-and-hold circuit which are both specifically developed for use in monitoring EEG during TMS stimulation.

Finally, controller 24 may communicate, for example, using a network interface or the like with a viewer of one or more medical imaging systems 29, including but not limited to a magnetic resonance imaging (MRI) machine, an x-ray computed tomography (CT) machine and the like, to receive information about the location of a lesion or other injury in the brain of the patient 18 detectable by that imaging modality. This information can be registered to the position information from the position processor 21, for example, through the use of common fiducial points as is understood in the art, to provide a common reference coordinate system.

Generally, as will now be described, the controller 24 will coordinate operation of the TMS device 12 and the EEG processor 20 to stimulate the brain of the patient 18 and to acquire EEG data after that stimulation, the EEG data linked to location information of the stimulation and registered with respect to the time of the stimulating signal through the coil 16.

Referring now to FIG. 2, in the first step of assessment of brain injury, the stored program 25 of the controller 24 may operate to receive information about a brain lesion or injury from a medical imaging system 29 as discussed above, or alternatively, this information may be entered by the user through the terminal 27 and as indicated by process block 30.

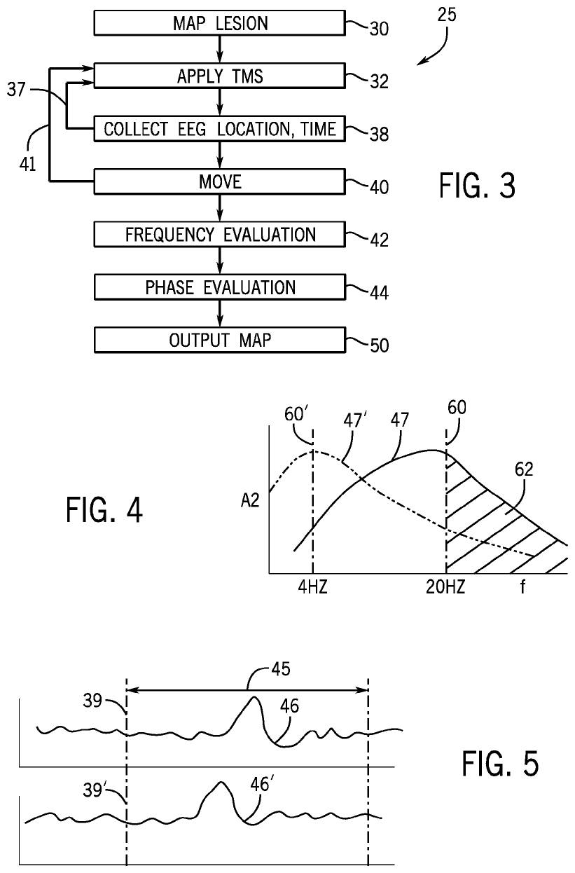

As indicated by process block 32, and referring also to FIG. 3, the program 25 may implement a scanning process in which the coil 16 is moved to different locations on the head of the patient 18 so that TMS probing stimulations can be applied to the patient 18 at a number of locations 34 positioned around the site of injury or lesion 36 in a regular scanning pattern or selectively as needed. This pattern may be implemented, as noted above, by instructions to a health care provider who may manually locate the coil 16, for example, under the guidance of a display screen on the terminal 27 or the like. Alternatively, this pattern may be implemented by signals provided to a robotic manipulator holding the coil 16 as described above.

At each location 34, one or several TMS pulses may be applied through the coil 16 and then as indicated by process block 38, EEG data collected through EEG processor 20 and linked to the particular location 34 and the time of application of the stimulating pulse.

As indicated by arrow 37, at a given location 34, a second TMS pulse may be then applied to the patient 18 and, again, corresponding EEG data collected through EEG processor 20, this data also linked to the particular location 34 and the time of the application of the stimulation pulse so that multiple sets of data are obtained at each location 34.

After this repeated measures of EEG data are collected for a given stimulation 39, at process block 40, the coil 16 is moved and process blocks 32 and 38 repeated per arrow 41 (including the multiple acquisitions per arrow 37) until a complete data set of locations 34 around the injury or lesion 36 has been collected for each of the locations 34.

Referring now to FIGS. 2 and 3, as indicated by process block 42, the collected data is then characterized in the frequency domain to detect cortical bistability. In this analysis, and referring to FIG. 4, EEG data is analyzed in a window after the time of stimulation (for example, 500 ms after the stimulation time) with respect to its spectral energy. Spectral energy can be obtained from the power spectrum 47 of the Fourier or Wavelet transform of the EEG signal within this window. Generally, this spectral measurement provides a quantification of the level of activity induced and/or evoked by the brain stimulation. In the case of cortical bistability, neurons react briefly to the external brain stimulation and then fall or transition into an OFF-period, thereby interrupting a causal chain of neural interactions.

For each location 34, the measurement at process block 42 may be made after first normalizing the power spectrum 47 to an average power spectrum obtained from EEG signals in a window before the time of brain stimulation (for example, from 500 ms before stimulation time). After this normalization, the strongest power modulation points 60 induced by the brain stimulation over time is computed. This is the natural frequency measurement. The result is a quantitative measurement of the frequency content of the response, whereby a low frequency activity indicates the presence of a slow wave. Then, power above 20 Hz represented by shaded area 62 is accumulated to assess the presence of a significant suppression of neuronal firing indicative of an OFF-period in the case of cortical bistability. This is the strength of high-frequency activity measurement.

Referring now to FIGS. 2 and 5, at succeeding process block 44, a time domain coherence of the EEG signals 46 and 46′ in corresponding windows 45 after each of multiple (e.g. 100) stimulation pulses 39 and 39′ for each given location 34 is assessed. The window 45 may match that described above with respect to the EEG signals used for the power spectrum measurement, and this time domain assessment is used to establish whether the neural activity is correlated to the stimulation pulses 39. In one embodiment, the time domain assessment identifies the phase of the signals 46 or 46′ (e.g., a dominant phase with respect to signal power), for example, by Fourier or Wavelet transform of the signals 46 or 46′ with respect to a time of the corresponding stimulation pulses 39 and 39′. Strong phase coherence occurs when those phase measurements cluster.

One method of quantifying phase coherence establishes an inter-trial coherence that adds normalized vectors of each phase of the signals 46 or 46′ together to provide a measure equal to the magnitude of the vector sum of these phases. Alternative methods of establishing coherence may, for example, look at correlations between the EEG signals 46 and 46′ (associated with the different stimulation pulses 39 and 39′) to similar effect.

These measurements of phase coherence may be normalized to the phase coherence of EEG data outside of the window 45, for example, at times before the stimulation 39 or 39′. In one example, the phase coherence for this normalization process may be characterized using inter-trial coherence of the EEG signals 46 and 46′ during those times outside of the windows 44.

These measurements provide a quantitative measurement characterizing causality between the stimulation pulses 39 and 39′ and the signals detected in EEG signals 46 and 46′ in the windows 44.

At process block 50, the natural frequency, the strength of high frequency activity suppression, and the duration of phase-locking factor are combined into a multivariate statistical index aimed at scoring the degree of alteration of local cortical functionality. Specifically, a partial least square regression model can be applied on the above-mentioned features to perform multivariate analysis; regression weights can be calibrated on a benchmark dataset including both normotypical healthy subjects as well as brain-injured patients stratified by clinical standardized disability scales.

Referring again to FIGS. 2 and 3, at process block 50, the resulting index value may be output on the display of terminal 27, for example, on an outline of a top-plan view or top flattened view of the surface of the brain with shaded regions 52 indicating abnormally high bistability. Shading may be implemented as a pixel luminance, color, or the like in proportion to the index value. A quantitative value 54 may also be provided for longitudinal studies as well.

Certain terminology is used herein for purposes of reference only, and thus is not intended to be limiting. For example, terms such as “upper”, “lower”, “above”, and “below” refer to directions in the drawings to which reference is made. Terms such as “front”, “back”, “rear”, “bottom” and “side”, describe the orientation of portions of the component within a consistent but arbitrary frame of reference which is made clear by reference to the text and the associated drawings describing the component under discussion. Such terminology may include the words specifically mentioned above, derivatives thereof, and words of similar import. Similarly, the terms “first”, “second” and other such numerical terms referring to structures do not imply a sequence or order unless clearly indicated by the context.

When introducing elements or features of the present disclosure and the exemplary embodiments, the articles “a”, “an”, “the” and “said” are intended to mean that there are one or more of such elements or features. The terms “comprising”, “including” and “having” are intended to be inclusive and mean that there may be additional elements or features other than those specifically noted. It is further to be understood that the method steps, processes, and operations described herein are not to be construed as necessarily requiring their performance in the particular order discussed or illustrated, unless specifically identified as an order of performance. It is also to be understood that additional or alternative steps may be employed.

References to “a controller” and “a processor” or “the microprocessor” and “the processor,” can be understood to include one or more electronic computer processors that can communicate in a stand-alone and/or a distributed environment(s), and can thus be configured to communicate via wired or wireless communications with other processors for devices using standard electrical interfaces and protocols, where such one or more processor can be configured to operate on one or more processor-controlled devices that can be similar or different devices. Furthermore, references to memory, unless otherwise specified, can include one or more processor-readable and accessible memory elements and/or components that can be internal to the processor-controlled device, external to the processor-controlled device, and can be accessed via a wired or wireless network.

It is specifically intended that the present invention not be limited to the embodiments and illustrations contained herein and the claims should be understood to include modified forms of those embodiments including portions of the embodiments and combinations of elements of different embodiments as come within the scope of the following claims. All of the publications described herein, including patents and non-patent publications, are hereby incorporated herein by reference in their entireties

To aid the Patent Office and any readers of any patent issued on this application in interpreting the claims appended hereto, applicants wish to note that they do not intend any of the appended claims or claim elements to invoke 35 U.S.C. 112(f) unless the words “means for” or “step for” are explicitly used in the particular claim.

I claim:

1. An apparatus for assessing brain injury comprising:
an electroencephalographic sensor system collecting electrical signals from electrically active neurons and neural circuits within a patient's brain;
a neural stimulator for triggering a localized stimulation of neurons within the patient's brain at a set of different location and times using electrical, magnetic, or ultrasonic stimulation; and
an electronic computer receiving the electrical signals from the electroencephalographic sensor and executing a stored program to:
(i) monitor electrical signals from the electroencephalographic sensor in response to localized stimulations at the different locations and times from firing neurons and active neural circuits;
(ii) determine a cortical bistability value at the different locations and times; and
(iii) output a map providing information about cortical bistability by linking the determined cortical bistability to different neocortical regions according to the different locations.

2. The apparatus of claim 1 further including a position tracker monitoring a location of the neural stimulator to provide a location value of the localized stimulation to the electronic computer and wherein the location of value is linked to a corresponding electrical signal and determination of cortical bistability to provide the map.

3. The apparatus of claim 1 wherein the electronic computer further executes the stored program to receive physiological injury data locating an injury site in the brain and wherein the map provides an indication of the injury site.

4. The apparatus of claim 1 wherein the electronic computer determines cortical bistability by determining high-frequency activity, wherein reduced high-frequency activity is indicative of increased cortical bistability.

5. The apparatus of claim 4 wherein the determination of high-frequency activity evaluates spectral energy above a predetermined threshold.

6. The apparatus of claim 4 wherein the electronic computer further determines cortical bistability by assessing broad-band spectral activity induced by localized stimulation, wherein reduced spectral peak power is indicative of increased cortical bistability.

7. The apparatus of claim 6 wherein the determination of broad-band spectral activity evaluates a peak spectral power.

8. The apparatus of claim 4 wherein the electronic computer further determines cortical bistability by assessing a phase relationship between the electrical signals and a time of localized stimulation of neurons associated with electrical signals.

9. The apparatus of claim 8 wherein the phase relationship is evaluated in electrical signals band limited to more than 4 Hz and less than 40 Hz.

10. The apparatus of claim 8 wherein the phase relationship is established by evaluating an inter-trial coherence in electrical signals associated with two different times of localized stimulation of neurons.

11. The apparatus of claim 1 wherein the determination of cortical bistability values is determined using the electrical signals after the time of localized stimulation only in a time window of less than 500 ms after the localized stimulation.

12. The apparatus of claim 1 wherein the electroencephalographic sensor employs cutaneous electrodes placed on the patient and the neural stimulator is a transcranial magnetic stimulation device.

13. The apparatus of claim 1 wherein the map may be an image representation of the brain with regions of cortical bistability having predetermined shading corresponding to the degree of bistability.
